(19) United States
(12) Patent Application Publication
(10) Pub. No.: US 2013/0065812 A1
(43) Pub. Date: Mar. 14, 2013

(54) WATER-DILUTABLE SOLID CONCENTRATE

(71) Applicants: Timothy J. Kohnke, Ogden, IA (US); Mark D. Levitt, Lake Elmo, MN (US); Amanda R. Blattner, Prior Lake, MN (US); Charles A. Hodge, Cottage Grove, MN (US)

(72) Inventors: Timothy J. Kohnke, Ogden, IA (US); Mark D. Levitt, Lake Elmo, MN (US); Amanda R. Blattner, Prior Lake, MN (US); Charles A. Hodge, Cottage Grove, MN (US)

(73) Assignee: Ecolab USA Inc., St. Paul, MN (US)

(21) Appl. No.: 13/232,619

(22) Filed: Sep. 14, 2011

(51) Int. Cl. C11D 17/00; C11D 17/08; B29C 39/02; A61L 9/012

(52) U.S. Cl. 510/437; 512/4; 512/1; 510/481; 264/299

(57) ABSTRACT

A solid composition for cleaning or fragrancing includes at least one ethoxylated alcohol, at least one C12-C24 saturated fatty acid sodium salt, water, at least one glycol, and at least one water insoluble component. The solid composition can have a weight ratio of water to glycol to C12-C24 saturated fatty acid sodium salt between about 2:2.5:1 and about 3:4:1.

### TECHNICAL FIELD

The present invention is related to the field of water-dilutable solids and use solutions made from such solids. In particular, the present invention is related to a water-dilutable solid concentrate containing a surfactant and a water insoluble component.

### BACKGROUND

Water-dilutable solid products are used in an array of applications and can be converted to a concentrate solution or a use solution by dissolving the solid in water. Example solutions include fragrance or air-freshener solutions and cleaning solutions.

Water-dilutable solid air-freshener products can be diluted to a final use solution using a dispensing apparatus. One type of dispensing apparatus includes a sump which sprays water onto an exposed surface of the solid to dissolve a portion of the composition as a pre-dilution before being diluted to a final concentration. Another type of air-freshening dispensing apparatus mixes the solid and water to a final concentration in one step.

One challenge that arises when transporting and storing water-dilutable solid products is that the solid can be exposed to higher temperatures. The result is that the water-dilutable solid can weep or melt, making the product later unpleasant to handle and perhaps unsuitable for the dispensing machine.

The amount of water required to dissolve and dispense the solid in a given period of time may also be of concern. Water-dilutable solid products frequently contain a water insoluble component, and, as described above, water may be sprayed or impinged upon an exposed surface of the solid to dissolve a portion of the composition. Previously the dilution was limited by the solubility of the water insoluble component. To meet dispense rates, it becomes desirable to increase the solubility of the solid product and maximize the concentration of the water insoluble component in the solid. Increasing the water insoluble component concentration reduces the amount of water-dilutable solid product that must be dissolved in a given time period to deliver the required level of water insoluble component in the use solution. In addition, increasing the water insoluble component concentration increases the amount of components present in the water-dilutable solid which provides value to the end user.

### SUMMARY

The present invention includes a solid composition for cleaning or fragrancing. The solid composition includes at least one ethoxylated alcohol, at least one C12-C24 saturated fatty acid sodium salt, water, at least one glycol, and at least one water insoluble component.

In one embodiment, the present invention is a solid composition having a water to glycol to C12-C24 saturated fatty acid sodium salt weight ratio between about 2:2.5:1 and about 3:4:1. The water insoluble component can be selected from a group consisting of fragrance oils, hydrocarbon solvents, glycol ethers, ester solutions, solvents, biocides, defoamers, lubricants, rinse additives and polishes.

In yet another embodiment, the solid composition is used in a use solution, in which the solid is mixed with dilution water. The solid can be mixed with sufficient dilution water to form a use solution having a dilution ratio (calculated as a weight percentage) of at least about 1%.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIG. 1 is a graph showing the amount of solid product dispensed, in a fixed period of time, in grams per gallon of water dispensed versus changing water temperature.

FIG. 2 is a graph similar to FIG. 1 except at lower water temperatures.

FIG. 3 is a graph similar to FIG. 1 for a solid product having an alternative formulation.

### DETAILED DESCRIPTION

The present invention relates to a water-dilutable solid or a hydrate solid including at least one C12-C24 saturated fatty acid sodium salt, at least one glycol, water, at least one water insoluble component, and at least one ethoxylated alcohol. In one example, the water-dilutable solid consists essentially of at least one ethoxylated alcohol, at least one C12-C24 saturated fatty acid sodium salt, water, hexylene glycol and at least one fragrance oil. In general a water-dilutable solid refers to a non-flowable composition that dissolves in water. The water-dilutable solid can have a relatively high water insoluble component concentration while being stable at increased temperatures and having a minimum dissolution time with minimal dilution water. The water-dilutable solid can be used in any environment in which it is desirable to form a solution having a high insoluble component concentration. For example, the water-dilutable solid can be used in institutional, vehicle care, quick service restaurants, and textile care and laundering applications. Such applications include but are not limited to laundry and textile cleaning and destaining, vehicle cleaning and care applications, surface cleaning and destaining, kitchen and bath cleaning and staining, floor cleaning and destaining, general purpose cleaning and destaining, industrial and household cleaners, and air-freshening systems.

The water-dilutable solid contains at least one C12-C24 saturated fatty acid sodium salt. The C12-C24 saturated fatty acid sodium salt functions as a binder that holds the water-dilutable solid composition together. A suitable concentration of C12-C24 saturated fatty acid sodium salt in the water-dilutable solid is between approximately 1% and approximately 15% by weight, and more preferably between approximately 4% and about 10% by weight. Suitable C12-C24 saturated fatty acid sodium salts include C14-C22 saturated fatty acid sodium salts, more suitably C16-C20 saturated fatty acid sodium salts. Suitable saturated fatty acid sodium salts also include blends of saturated fatty acid sodium salts that include C18-C24 saturated fatty acid sodium salts, and more particularly C18 saturated fatty acid sodium salts. An example of commercially available suitable C12-C24 saturated fatty acid sodium salts includes sodium stearate. Sodium stearate may be commercially provided as C18 saturated fatty acid sodium salts blended with saturated fatty acid sodium salts of other carbon chain lengths. For example, a commercially available sodium stearate may predominantly contain a blend of C16 and C18 saturated fatty acid sodium salts. One commercially available sodium stearate is sodium stearate T-1 available from Chemtura Corporation, Middlebury, Conn.

The water-dilutable solid also contains at least one glycol. The glycol is a solvent that adjusts the polarity of the liquid phase (i.e., the water, solvent, water insoluble component, such as fragrance oil, and ethoxylated alcohol). Suitable glycols include but are not limited to hexylene glycol and propylene glycol. A suitable concentration of glycol in the water-dilutable solid is between approximately 3% and approximately 35% by weight. A particularly suitable concentration of glycol is between approximately 12% and approximately 30% by weight.

The water-dilutable solid further contains water. Water may be independently added to the composition or may be provided in the composition as part of its presence in an aqueous material that is added to the detergent composition. For example, materials added to the solid composition may include water or may be prepared in an aqueous premix. Typically, water is introduced into the composition to provide a desired viscosity for processing prior to solidification and to provide a desired rate of solidification. The water may also disperse and dissolve the C12-C24 saturated fatty acid sodium salts. For example, not enough water can result in the components staying in powder form so that a suitable solid cannot be formed. The water may be provided ion-free, sourced from: distilled, de-ionized (DI), reverse osmosis (RO); or as softened water. A suitable component concentration includes between about 1% and about 25% by weight water, and particularly between about 7% and about 20% by weight water.

A suitable water to glycol to C12-C24 saturated fatty acid sodium salt weight ratio is between approximately 1.3:3.6:1 and approximately 3:4:1. A particularly suitable water to glycol to C12-C24 saturated fatty acid sodium salt weight ratio is between 2:2.5:1 and approximately 3:4:1. A more particularly suitable water to glycol to C12-C24 saturated fatty acid sodium salt weight ratio is between approximately 2:3:1 and approximately 3:4:1.

The water-dilutable solid still further contains at least one water insoluble component. The water insoluble component can be any organic component that is insoluble or is minimally soluble in water. A suitable water insoluble component can be less than 10% soluble in 20° C. water. A particularly suitable water insoluble component can be less than 5% soluble in 20° C. water. A more particularly suitable water insoluble component can be less than 2% soluble in 20° C. water.

Example water insoluble components include, but are not limited to, fragrance oils or oil based fragrances, hydrocarbon solvents, glycol ethers, ester solvents, solvents, biocides, defoamers, lubricants, rinse additives, and polishes. Additional example water insoluble components include, but are not limited to, d-limonene, methyl laurate, diethyl succinate, tripropylene glycol n-butyl ether, silicone oil, and ortho phenyl phenol. Examples of particularly suitable commercially available water insoluble components include fragrance oil mixtures or solutions formed from one or more oils, which may each have a different degree of hydrophobicity. Examples of more particularly suitable commercially available water insoluble components include citrus basil apple fragrance, orange ginger fragrance and lavender paradise fragrance, each available from Arylessence, Marietta, Ga., and fragrance US251225/00 available from Givaudan Fragrance Company, Teaneck, N.J.

The water-dilutable solid comprises one or more water insoluble components. A suitable concentration of water insoluble components in the water-dilutable solid is between approximately 1% and approximately 30% by weight. A particularly suitable concentration of water insoluble components in the water-dilutable solid is between approximately 5% and approximately 25% by weight. A more particularly suitable concentration of water insoluble components in the water-dilutable solid is between approximately 10% and approximately 20% by weight.

The water-dilutable solid also includes at least one ethoxylated alcohol. The ethoxylated alcohol is chosen to improve the solubility of the water insoluble components. Suitable ethoxylated alcohols include ethoxylated C10-C18 alcohols. Particularly suitable ethoxylated alcohols include ethoxylated C10-C15 alcohols, and more particularly suitable ethoxylated alcohols include ethoxylated C9-C11 alcohols. Example suitable commercially available ethoxylated alcohols include, but are not limited to: Tomadol 91-8, Tomadol 25-12 and Tomadol 25-7, which are each available from Air Products and Chemicals, Inc., Allentown, Pa.

The water-dilutable solid can include more than one ethoxylated alcohol depending upon the solubility demands from the non-soluble components. In one embodiment, the water-dilutable solid can include at least two ethoxylated alcohols. For example, the water-dilutable solid can include a first ethoxylated C10-C15 alcohol and a second ethoxylated C10-C15 alcohol. In another example, the water-dilutable solid includes a C10-C15 ethoxylated alcohol having about 12 ethylene oxide groups and a C10-C15 ethoxylated alcohol having about 7 ethylene oxide groups.

A suitable ethoxylated alcohol concentration (i.e., total ethoxylated alcohol concentration) of the water-dilutable solid is between approximately 30% and approximately 60% by weight. When two or more ethoxylated alcohols are present, they may be present in equal or unequal amounts.

The water-dilutable solid can be formed by casting, pressing or extruding methods. In one suitable method, the water-dilutable solid is formed by mixing at least one C12-C24 saturated fatty acid sodium salt, water, at least one glycol and at least one ethoxylated alcohol to form a mixture. These components may be referred to as solidification components. The mixture is heated and stirred until it forms a clear and uniform solution. The solution is then cooled and at least one water insoluble component is added. Once uniform, the mixture is poured into a suitable mold and hardened by further cooling. In a particularly suitable method, the C12-C24 saturated fatty acid sodium salt, water, glycol and ethoxylated alcohol solution is heated to 90° C., mixed, and then is cooled to about 80° C. Next, at least one water insoluble component is added, and the mixture is poured into a suitable mold.

By the term “solid”, it is meant that the hardened composition will not flow and will substantially retain its shape under moderate stress, strain, pressure or mere gravity. More specifically, the hardened composition is non-compliant and non-formable as compared to a gel, which is compliant and formable. The degree of hardness of the solid cast composition may range from that of a fused solid product which is relative dense and hard, for example, like concrete, to a consistency characterized as being a hardened paste. In addition, the term “solid” refers to the state of the hardened composition under the expected conditions of storage and use. In general, it is expected that the water-dilutable solid will be a stable solid that remains in solid form when exposed to temperatures of up to approximately 38° C. (100° F.) and particularly greater than approximately 49° C. (120° F.). If a water-dilutable solid excessively weeps or melts after solidification, various problems may occur, including but not limited to: decreased integrity, appearance and inability to dispense or package the solid. In one example, the water-dilutable solid does not excessively melt or weep when stored at 50° C. (122° F.) for about one week. In another example, the water-dilutable solid contains at least about 15% by weight water insoluble component and does not excessively melt or weep when stored at 50° C. (122° F.) for about one week.

The water-dilutable solid is a concentrate solid which can be diluted with water, known as dilution water, to form a concentrate solution or a use solution. In general, a concentrate refers to a composition that is intended to be diluted with water to provide a use solution; a use solution is dispersed or used without further dilution. Example concentrate solutions and use solutions include cleaning solutions and solutions for use with air-freshening systems.

A use solution can be prepared by dissolving the water-dilutable solid with water at a dilution ratio that provides the desired water insoluble component concentration. The water-dilutable solid is soluble at the desired dilution ratio. In one example, the water-dilutable solid completely dissolves when mixed with water having a temperature between about 10° C. and about 38° C. (50° F. to 100° F.) and a clear uniform solution is formed. Alternatively, the water-dilutable solid may form a stable emulsion or dispersion in water. In one example, the water-dilutable solid forms a microemulsion when mixed with dilution water between about 10° C. to about 38° C. (50° F. to 100° F.).

Suspended solids, which may cause cloudiness or haziness in the use solution, can be measured with a nephelometer using EPA method 180.1. In one example, the water-dilutable solid dissolves when mixed with the dilution water to form a use solution having a turbidity of less than about 50 Nephelometric Turbidity Units (NTU). In another example the water-dilutable solid dissolves when mixed with the dilution water to form a use solution having a turbidity of less than about 20 NTU and more suitably less than about 5 NTU. The dilution water may be heated to improve the rate of dissolution. In one example, diluting the water-dilutable solid at at least a 1% dilution ratio (i.e., dilution water:water-dilutable solid weight ratio of 100:1) with dilution water having temperature between 10° C. to about 38° C. (50° F. to 100° F.) forms a clear use solution in which the water-dilutable solid completely dissolves.

The resulting concentrate solution or use solution has a relatively high water insoluble component concentration. In one suitable solution, the water insoluble component concentration is greater than 0.2%. In a particularly suitable solution, the water insoluble component concentration is greater than 0.3%. In a more particularly suitable solution, the water insoluble component concentration is greater than 0.4%. The relatively high water insoluble component concentration requires less dilution water when forming the use solution.

Additionally, the saturation concentration of the water insoluble component may be higher in the use solution than in water at the same conditions, such as temperature and pressure. The increased solubility of the water insoluble component may be due to the ethoxylated alcohols forming a solublizing system. In one example, the water-dilutable solid is completely soluble in water although the water insoluble component is present in an amount greater than the saturation concentration of the component in water at the same conditions.

It was surprisingly found that the water-dilutable solid completely dissolves to form a low viscosity use solution with minimal dilution water. The viscosity can be measured with a Brookfield LVDV-II viscometer using the LV-1 spindle, at 100 rmp and room temperature. One suitable water-dilutable solid completely dissolves in about 10° C. to about 49° C. (50° F. to 120° F.) water to form a low viscosity, free flowing liquid having a viscosity less than about 100 centipoise. A particularly suitable water-dilutable solid completely dissolves in about 10° C. to about 49° C. (50° F. to 120° F.) water to form a low viscosity, free flowing liquid having a viscosity less than about 10 centipoise. Another suitable water-dilutable solid completely dissolves in the dilution water to form a use solution having a dilution percentage of at least 1%.

The water-dilutable solid can be diluted to the use solution in one step. Alternatively, the water-dilutable solid can be diluted to form a pre-dilution solution, which is diluted to a final use solution concentration in a second dilution step. An example of a two-step dilution is an air freshening system which pre-dilutes the water-dilutable solid in a sump before forming the final concentration. One suitable water-dilutable solid completely dissolves when diluted with water in a water-dilutable solid to water weight ratio containing up to about 85% water-dilutable solid by weight.

### EXAMPLES

The present invention is more particularly described in the following examples that are intended as illustrations only, since numerous modifications and variations within the scope of the present invention will be apparent to those of skill in the art. Unless otherwise noted, all parts, percentages, and ratios reported in the following examples are on a weight basis, and all reagents used in the examples were obtained or are available from the chemical suppliers described below or may be synthesized by conventional techniques.

### Materials Used

Tomadol 25-12: a nonionic surfactant made from linear C12-C15 alcohol with 11.9 moles (average) of ethylene oxide groups, available from Air Products and Chemicals, Inc., Allentown, Pa.

Tomadol 25-7: a nonionic surfactant made from linear C12-C15 alcohol with 7.3 moles (average) of ethylene oxide groups, available from Air Products and Chemicals, Inc., Allentown, Pa.

Tomadol 91-8: a nonionic surfactant made from linear C9-C11 alcohol with 8.3 moles (average) of ethylene oxide groups, available from Air Products and Chemicals, Inc., Allentown, Pa.

Sodium stearate T-1: a sodium stearate blend available from Chemtura Corporation, Middlebury, Conn.

Fragrance US251225/00: a mixture of fragrance oils available from Givaudan Fragrance Company, Teaneck, N.J.

Citrus Basil Apple Fragrance: a mixture of fragrance oils available from Arylessence, Marietta, Ga.

Orange Ginger Fragrance: a mixture of fragrance oils available from Arylessence, Marietta, Ga.

Millithix® 925S: a dibenzylidene sorbitol available from Milliken Chemical, Spartanburg, S.C.

Neodol 25-12: alcohol ethoxylate available from Shell Chemical Co., Houston, Tex.

Neodol 25-7: alcohol ethoxylate available from Shell Chemical Co., Houston, Tex.

Ethox HVB: a PEG-175 diisostearate available from Ethox Chemicals, Greenville, S.C.

Ethox P 6000 DS SP: a PEG-150 distearate available from Ethox Chemicals, Greenville, S.C.

Ethox MS-23: a POE (23) stearate available from Ethox Chemicals, Greenville, S.C.

Sylvaclear® PA1200 and Sylvaclear® PE 400V: polyamide resins available from Arizona Chemical, Jacksonville, Fla.

Versamid® 744, Versamid® 930, Versamid® 940, and Versamid® 1655: polyamide resin available from Cognis, Monheim, Germany

Lavender Paradise: a mixture of fragrance oils available from Arylessence, Marietta, Ga.

### Examples 1-5 and Comparative Example A

The raw materials identified for each of Example 1-5 in Table 1 below were combined and mixed. Each of Examples 1-5 contained Tomadol 25-7, Tomadol 25-12, a mixture of fragrance oils, and a select binder. The binder was varied for Examples 1-5. Comparative Example A did not include Tomadol 25-7 or Tomadol 25-12. That is, Comparative Example A did not include an ethoxylated alcohol.

TABLE 1

| | | | | | | Comp. |
|---|---|---|---|---|---|---|
| | Ex. 1 | Ex. 2 | Ex. 3 | Ex. 4 | Ex. 5 | Ex. A |
| Tomadol 25-12(wt %) | 36.6% | 34.5% | 30.2% | 34.6% | 22.6% | |
| Tomadol 25-7(wt %) | 14.1% | 11.9% | 15.1% | 18% | 8.7% | |
| Orange ginger fragrance (wt %) | | 23.5% | 20.3% | | | |
| Hexylene glycol (wt %) | 15% | | | | | 15% |
| Water (wt %) | 10% | | | | | 60.7% |
| Sodium stearate T-1 (wt %) | 5% | | | | | 5% |
| PEG 8000 (wt %) | | 30% | | | | |
| PEG 4000 (wt %) | | | 34.4% | | | |
| Urea(wt %) | | | | | 57% | |
| Stearyl alcohol (wt %) | | | | 17% | | |
| Propylene glycol (wt %) | | | | 10.4% | | |
| Citrus basil apple fragrance (wt %) | 19.28% | | | 20% | 11.7% | 19.28% |

All raw materials except the fragrance were mixed to form a mixture and then heated to 90° C. and stirred to form a clear, uniform solution. The solution was then cooled to 80° C. and the fragrance was added. Once uniform, the mixture was poured into a suitable mold and hardened by further cooling to form a solid product.

An oven stability test was conducted in which the solids were placed in an oven at 50° C. for seven days to determine the stability of the solids. Visual observations of the solids following the storage period were recorded.

The solids were also diluted with dilution water to form a use solution. The dilution test investigated the water temperature needed to dispense and form a use solution having 0.15% to approximately 0.31% by weight fragrance, within a given period of time. Observations regarding the use solution and the dilution time were recorded. Table 2 provides the oven test results and the dilution test results.

TABLE 2

| | Comments |
|---|---|
| Ex. 1 | Solid product formed; 115° F. dilution water used to |
| | achieve at least 28 g/gal dispense rate |
| Ex. 2 | Solid product formed; 145° F. dilution water used to |
| | achieve at least 28 g/gal dispense rate |
| Ex. 3 | Solid product formed; 145° F. dilution water used to |
| | achieve at least 28 g/gal dispense rate |
| Ex. 4 | Unable to form a hard solid |
| Ex. 5 | Unable to form a hard solid |
| Comp. | Fragrance oils floated to surface, remained liquid, |
| Ex. A | no solid was formed |

Examples 1, 2 and 3 formed solid products. Of these examples, Example 1, which contained a sodium stearate binder, required the lowest water temperature to form a use solution meeting the desired dispense rate of at least 28 g/gal.

Control A, which did not include an ethoxylated alcohol, experienced fragrance oil floating to the surface and did not form a solid.

### Examples 6-26

Additional binders were also tried according to Examples 6-26 presented in Tables 3-6 below. These binders included potassium stearate, Millithix® 925S (Millithix), Sylvaclear PA1200, Sylvaclear PE 400V, Versamid 744, Versamid 930, Versamid 940 and Versamid 1655. All Experiments failed to form a solid product except Examples 11, 13 and 21-26. Example 11 formed a hard solid with chunks dispersed throughout, and Example 13 formed a hard opaque solid with chunks dispersed throughout. The results of Examples 21-26 are summarized in Table 7.

TABLE 3

| | | | | | | Citrus |
|---|---|---|---|---|---|---|
| | | Hexyl- | Potas- | | | basil |
| | DI | ene | sium | Tomadol | Tomadol | apple |
| | water | glycol | stearate | 25-12 | 25-7 | fragrance |
| | (wt %) | (wt %) | (wt %) | (wt %) | (wt %) | (wt %) |
| Ex. 6 | 10 | 15 | 5 | 36.63 | 14.09 | 19.28 |
| Ex. 7 | 8 | 12 | 5 | 39.25 | 15.08 | 20.66 |
| Ex. 8 | 6 | 9 | 5 | 41.88 | 16.08 | 22.04 |
| Ex. 9 | 4 | 6 | 5 | 44.48 | 17.1 | 23.42 |

TABLE 4

| | | | | | Citrus |
|---|---|---|---|---|---|
| | Propylene | | Neodol | Neodol | basil apple |
| | glycol | Millithix | 25-12 | 25-7 | fragrance |
| | (wt %) | (wt %) | (wt %) | (wt %) | (wt %) |
| Ex. 10 | 50 | 5 | 23.55 | 9.05 | 12.4 |
| Ex. 11 | 52.5 | 2.5 | 23.55 | 9.05 | 12.4 |
| Ex. 12 | 54 | 1 | 23.55 | 9.05 | 12.4 |
| Ex. 13 | 60 | 2 | 19.89 | 7.64 | 10.47 |

TABLE 5

| | Sylvaclear | Sylvaclear | Propylene | Tomadol | Tomadol | Citrus basil | Orange |
|---|---|---|---|---|---|---|---|
| | PA1200 | PE400V | glycol | 25-12 | 25-7 | apple fragrance | ginger fragrance |
| | (wt %) | (wt %) | (wt %) | (wt %) | (wt %) | (wt %) | (wt %) |
| Ex. 14 | 4.5 | 0 | 0 | 44 | 22 | 0 | 29.5 |
| Ex. 15 | 0 | 4.5 | 0 | 44 | 22 | 0 | 29.5 |
| Ex. 16 | 8.6 | 0 | 0 | 42.1 | 21.1 | 0 | 28.2 |
| Ex. 17 | 5 | 0 | 20 | 35.74 | 18.6 | 20.6 | 0 |
| Ex. 18 | 5 | 0 | 10 | 40.5 | 21.08 | 0 | 23.43 |
| Ex. 19 | 10 | 0 | 10 | 38.14 | 19.83 | 0 | 22.03 |
| Ex. 20 | 15 | 0 | 10 | 35.74 | 18.6 | 0 | 20.6 |

TABLE 6

| | | | | | Dipropylene | Tomadol | Tomadol | Citrus basil |
|---|---|---|---|---|---|---|---|---|
| | Versamid | Versamid | Versamid | Versamid | glycol | 25-12 | 25-7 | apple fragrance |
| | 744 | 930 | 940 | 1655 | (wt %) | (wt %) | (wt %) | (wt %) |
| Ex. 21 | 20 | 0 | 0 | 0 | 20 | 28.65 | 14.85 | 16.5 |
| Ex. 22 | 0 | 20 | 0 | 0 | 20 | 28.65 | 14.85 | 16.5 |
| Ex. 23 | 0 | 0 | 20 | 0 | 20 | 28.65 | 14.85 | 16.5 |
| Ex. 24 | 0 | 0 | 0 | 20 | 20 | 28.65 | 14.85 | 16.5 |
| Ex. 25 | 30 | 0 | 0 | 0 | 20 | 23.84 | 12.34 | 13.77 |
| Ex. 26 | 25 | 0 | 0 | 0 | 0 | 35.75 | 18.59 | 20.66 |

TABLE 7

| | Comments |
|---|---|
| Ex. 21 | Hard, dark brown solid formed that was less hard than Ex. 22. |
| | After 50 C. oven stability test, fragrance significantly weeps out |
| | of the block. |
| Ex. 22 | Brown, extremely hard solid formed; solid hardened faster than |
| | Ex. 21. Solid did not seem very water soluble. After 50 C. |
| | oven stability test, fragrance significantly weeps out of the |
| | block. |
| Ex. 23 | Brown solid formed similar to Ex. 22. Solid did not seem very |
| | water soluble. After 50 C. oven stability test, fragrance |
| | significantly weeps out of the block. |
| Ex. 24 | Hard, almost clear, solid formed. After 50 C. oven stability test, |
| | fragrance significantly weeps out of the block. |
| Ex. 25 | Hard solid formed. After 50 C. oven stability test, fragrance |
| | significantly weeps out of the block. |
| Ex. 26 | Hard solid formed. After 50 C. oven stability test, fragrance |
| | significantly weeps out of the block. A use solution formed with |
| | 0.31 wt % fragrance oil was very cloudy and white. |

### Examples 27-56

The water to solvent to sodium stearate ratio was adjusted in Examples 27-56, in which propylene glycol and hexylene glycol were used as solvents.

The raw materials identified for each of Examples 27-56 were mixed and formed into solids (when possible) according to the procedure for Examples 1-5. Visual observations of the concentrate solid products were recorded. Select solid products were placed in a 50° C. oven for seven days to determine the stability of the solid at an elevated temperature. Visual observations of the solids were recorded after the seven day storage period. The compositions of Examples 27-56 are presented in Table 8; the water:solvent:stearate weight ratios and observations are presented in Table 9.

TABLE 8

| | DI | Propylene | Hexylene | Sodium | Tomadol | Tomadol | Citrus basil |
|---|---|---|---|---|---|---|---|
| | water | glycol | glycol | stearate T-1 | 25-12 | 25-7 | apple fragrance |
| | (wt %) | (wt %) | (wt %) | (wt %) | (wt %) | (wt %) | (wt %) |
| Ex. 27 | 1 | 3 | 0 | 1 | 49.7 | 19.1 | 26.6 |
| Ex. 28 | 1.25 | 3.5 | 0 | 1 | 49.34 | 18.96 | 25.97 |
| Ex. 29 | 1.85 | 5.55 | 0 | 1.85 | 47.5 | 18.25 | 25 |
| Ex. 30 | 2.5 | 7 | 0 | 2 | 46.2 | 17.79 | 24.37 |
| Ex. 31 | 2.6 | 7.4 | 0 | 2 | 46.08 | 17.7 | 24.45 |
| Ex. 32 | 1.25 | 5.5 | 0 | 2.5 | 47.5 | 18.3 | 25 |
| Ex. 33 | 2.5 | 7.5 | 0 | 2.5 | 45.79 | 17.59 | 24.1 |
| Ex. 34 | 1 | 5.2 | 0 | 3 | 47.5 | 18.3 | 25 |
| Ex. 34 | 3 | 9 | 0 | 3 | 44.5 | 17.1 | 23.42 |
| Ex. 36 | 3.75 | 10.5 | 0 | 3 | 43.32 | 16.64 | 22.8 |
| Ex. 37 | 3.9 | 11.1 | 0 | 3 | 42.94 | 16.5 | 22.6 |
| Ex. 38 | 1 | 4.2 | 0 | 4 | 47.5 | 18.3 | 25 |
| Ex. 39 | 2 | 3.2 | 0 | 4 | 47.5 | 18.3 | 25 |
| Ex. 40 | 5 | 0 | 15.2 | 4 | 39.77 | 15.2 | 20.83 |
| Ex. 41 | 5 | 0 | 16.8 | 4 | 38.84 | 14.92 | 20.44 |
| Ex. 42 | 5 | 0 | 18 | 4 | 38.21 | 14.68 | 20.11 |
| Ex. 43 | 8 | 0 | 15.6 | 4 | 37.9 | 14.55 | 19.95 |
| Ex. 44 | 5 | 14 | 0 | 4 | 40.28 | 15.48 | 21.2 |
| Ex. 45 | 5.2 | 14.8 | 0 | 4 | 39.81 | 15.29 | 20.95 |
| Ex. 46 | 9 | 0 | 14.6 | 4 | 37.9 | 14.55 | 19.95 |
| Ex. 47 | 10 | 0 | 13.6 | 4 | 37.9 | 14.55 | 19.95 |
| Ex. 48 | 10 | 0 | 14.6 | 4.5 | 37.11 | 14.26 | 19.53 |
| Ex. 49 | 0 | 0 | 0 | 5 | 49.7 | 19.1 | 26.2 |
| Ex. 50 | 0 | 22.4 | 0 | 5 | 38 | 14.6 | 20 |
| Ex. 51 | 5.7 | 16.7 | 0 | 5 | 38 | 14.6 | 20 |
| Ex. 52 | 10 | 0 | 15 | 5 | 36.63 | 14.09 | 19.28 |
| Ex. 53 | 22.4 | 0 | 0 | 5 | 38 | 14.6 | 20 |
| Ex. 54 | 11.2 | 11.2 | 0 | 5 | 38 | 14.6 | 20 |
| Ex. 55 | 0 | 0 | 0 | 7.6 | 48.3 | 18.6 | 25.5 |
| Ex. 56 | 0 | 0 | 0 | 10 | 47.1 | 18.1 | 24.8 |

TABLE 9

| | Water:solvent:stearate | |
|---|---|---|
| | wt ratio | Comments |
| Ex. 27 | 1:3:1 | White solid with poor dispersion of stearate; no oven test |
| Ex. 28 | 1.3:3.5:1.0 | White mushy solid; no oven test |
| Ex. 29 | 1:3:1 | Semi-clear solid with white chunks dispersed; no oven test |
| Ex. 30 | 1.3:3.5:1 | Semi-clear solid with chunks dispersed; no oven test |
| Ex. 31 | 1.3:3.7:1 | Crystal clear solid; after 50 C. oven stability test solid had oil on the surface. |
| Ex. 32 | 0.5:2.2:1 | White solid with poor dispersion of stearate; no oven test |
| Ex. 33 | 1:3:1 | White solid with poor dispersion of stearate; no oven test |
| Ex. 34 | 0.3:1.7:1 | White solid with poor dispersion of stearate; no oven test |
| Ex. 34 | 1:3:1 | Mostly clear solid with some white chunks dispersed; no oven test |
| Ex. 36 | 1.3:3.5:1 | Semi-clear solid with chunks dispersed; no oven test |
| Ex. 37 | 1.3:3.7:1 | Crystal clear solid; after 50 C. oven stability test, solid had oil on the surface. |
| Ex. 38 | 0.3:1.1:1 | White solid with poor dispersion of stearate, hard to mix due to filming even |
| | | at high temp (90 C.); no oven test |
| Ex. 39 | 0.5:0.8:1 | Slightly white solid with trapped bubbles; no oven test |
| Ex. 40 | 1.3:3.8:1 | White solid with poor dispersion of stearate; no oven test |
| Ex. 41 | 1.3:4.2:1 | White solid with poor dispersion of stearate; no oven test |
| Ex. 42 | 1.3:4.5:1 | Semi-clear solid that formed a skin even at high temps (85 C.); no oven test |
| Ex. 43 | 2:3.9:1 | White solid with poor dispersion of stearate; no oven test |
| Ex. 44 | 1.3:3.5:1 | Solid with a few very small white chunks dispersed; no oven test |
| Ex. 45 | 1.3:3.7:1 | Crystal clear solid; after 50 C. oven stability test, solid had oil on the surface. |
| Ex. 46 | 2.3:3.7:1 | Clear solid; no oven test |
| Ex. 47 | 2.5:3.4:1 | Crystal clear solid; after 50 C. oven stability test, solid had oil on the surface. |
| Ex. 48 | 2.2:3.2:1 | Solid with a few very small white chunks dispersed; no oven test |
| Ex. 49 | 0:0:1 | Oil on surface, stearate did not disperse well, mushy mess; no oven test |
| Ex. 50 | 0:4.5:1 | Solid with a few very small white chunks dispersed; no oven test |
| Ex. 51 | 1.1:3.3:1 | Crystal clear solid; no oven test |
| Ex. 52 | 2:3:1 | Crystal clear solid; after 50 C. oven stability test, solid had oil on the surface. |
| Ex. 53 | 4.5:0:1 | Crystal clear solid; no oven test |
| Ex. 54 | 2.2:2.2:1 | Slightly white solid with trapped bubbles; no oven test |
| Ex. 55 | 0:0:1 | Oil on surface, stearate did not disperse well, mushy mess; no oven test |
| Ex. 56 | 0:0:1 | Oil on surface, stearate did not disperse well, mushy mess; no oven test |

### Examples 57-67

Examples 57-67 investigated the water to solvent to stearate ratio with a lavender paradise fragrance. The compositions of Examples 57-67 are presented in Table 10; the water:solvent:stearate weight ratios and observations are presented in Table 11.

TABLE 10

| | | Hexyl- | Sodium | | | Lavender |
|---|---|---|---|---|---|---|
| | DI | ene | stearate | Tomadol | Tomadol | paradise |
| | water | glycol | T-1 | 25-12 | 25-7 | fragrance |
| | (wt %) | (wt %) | (wt %) | (wt %) | (wt %) | (wt %) |
| Ex. 57 | 10 | 15 | 5 | 26.24 | 32.06 | 11.7 |
| Ex. 58 | 10 | 10 | 5 | 28.13 | 34.37 | 12.5 |
| Ex. 59 | 5 | 15 | 5 | 28.13 | 34.37 | 12.5 |
| Ex. 60 | 15 | 10 | 5 | 26.24 | 32.06 | 11.7 |
| Ex. 61 | 15 | 15 | 5 | 24.37 | 29.78 | 10.85 |
| Ex. 62 | 15 | 14 | 6 | 24.37 | 29.78 | 10.85 |
| Ex. 63 | 20 | 6 | 6 | 25.43 | 31.07 | 11.5 |
| Ex. 64 | 20 | 12 | 6 | 23.22 | 28.44 | 10.34 |
| Ex. 65 | 18 | 18 | 6 | 21.75 | 26.58 | 9.67 |
| Ex. 66 | 12 | 18 | 6 | 24 | 29.33 | 10.67 |
| Ex. 67 | 12.5 | 15 | 5 | 25.31 | 30.94 | 11.25 |

TABLE 11

| | Water:solvent:stearate | |
|---|---|---|
| | wt ratio | Comments |
| Ex. 57 | 2:3:1 | Solid formed with very few small white chunks dispersed. |
| | | Composition remained a solid with a very slight amount of |
| | | oil on the surface after the 50 C. oven stability test. |
| Ex. 58 | 2:2:1 | Solid formed with a few small white chunks dispersed; |
| | | chunks were worse than Ex. 57. Composition remained a |
| | | solid with a very slight amount of oil on the surface after |
| | | the 50 C. oven stability test. |
| Ex. 59 | 1:3:1 | Most stearate not dissolved and a soft paste, not a solid, was |
| | | formed. |
| Ex. 60 | 3:2:1 | Mostly clear solid formed with only a few white chunks. |
| | | Chunks were better than Ex. 57. After 50 C. oven stability |
| | | test, composition was a liquid. |
| Ex. 61 | 3:3:1 | Stayed liquid for a long time when cooling. Eventually the |
| | | composition turned solid, but it was liquid after 50 C. oven |
| | | stability test. |
| Ex. 62 | 2.5:2.3:1 | Mostly clear solid formed with significant amount of white |
| | | chunks dispersed. No oven stability test. |
| Ex. 63 | 3.3:1:1 | Clear solid formed. Stayed liquid for a long time when |
| | | cooling. Eventually turned solid, but was liquid after the |
| | | 50 C. oven stability test. |
| Ex. 64 | 3.3:2:1 | Clear solid formed. Stayed liquid for a long time when |
| | | cooling. Eventually turned solid, but was liquid after the |
| | | 50 C. oven stability test. |
| Ex. 65 | 3:3:1 | Clear solid formed. Stayed liquid for a long time when |
| | | cooling. Eventually turned solid, but was liquid after the |
| | | 50 C. oven stability test. |
| Ex. 66 | 2:3:1 | Hard solid formed with white chunks dispersed. After 50 C. |
| | | oven stability test the composition was solid with some oil |
| | | on the surface. |
| Ex. 67 | 2.5:3:1 | Clear solid formed with a few white chunks dispersed. The |
| | | composition was a thick liquid after the 50 C. oven stability |
| | | test. |

### Example 68

The raw materials identified in Table 12 were mixed and formed into a solid product as described above with respect to Examples 1-5.

TABLE 12

| | Ex. 68 |
|---|---|
| De-ionized water (wt %) | 10% |
| Hexylene glycol (wt %) | 13.65% |
| Sodium stearate T-1 (wt %) | 4% |
| Tomadol 25-12 (wt %) | 37.9% |
| Tomadol 25-7 (wt %) | 14.55% |
| Citrus basil apple fragrance oil (wt %) | 19.95% |

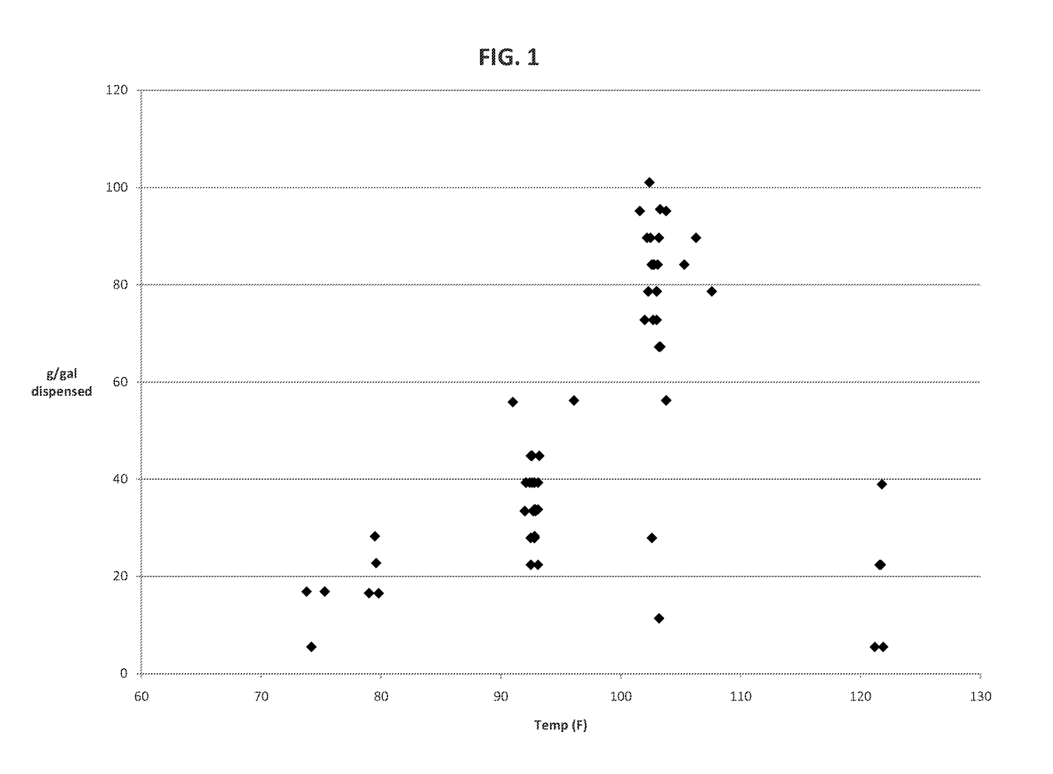

The solid product of Example 68 was tested in a load cell spray device and the water spray temperature was adjusted. A load cell spray device sprays the solid product with water to dissolve a portion of the solid and form a use solution. The solid product was loaded into a capsule, and the capsule was placed on a platform such that the solid product was exposed at the bottom of the capsule. The platform was connected to a load cell that tracked the mass of the solid block. Water was sprayed onto an exposed surface of the solid block with a nozzle using a spray time of 15 seconds, a dwell (rest) time of 2 minutes, and a flow rate of 1.16 gallons per minute (gpm) (4400 milliliters per minute). The nozzle was a yellow, glass-filled polypropylene nozzle commercially available from Spray Systems Co. having a 3.9 gpm flow rate at 10 psi and 84 degree spray angle. The nozzle was positioned 1-1.5 inches from the solid product. The weight loss of the solid product was measured after each spray and compared to the volume of water sprayed to determine the grams of solid per gallon dispensed. The test results are presented in the graph of FIG. 1 which plots the grams per gallon (g/gal) dispensed versus the temperature of the sprayed water in degrees Fahrenheit.

### Example 69

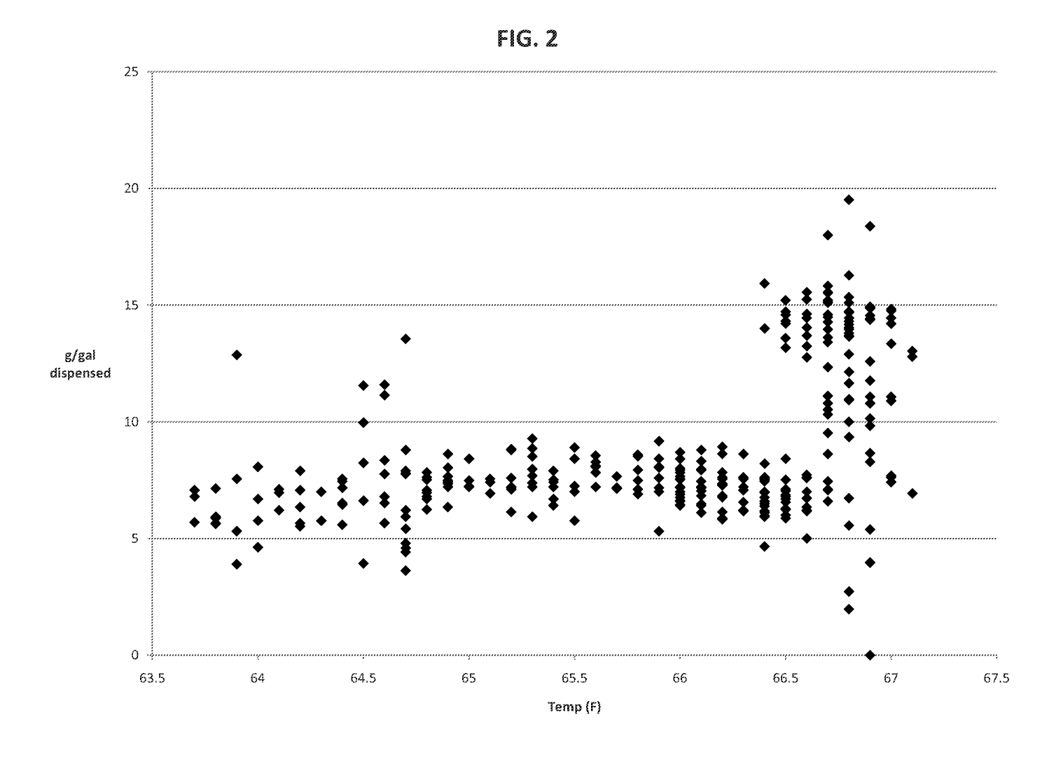

The solid product having the same composition as Example 68 was also tested in the load cell spray device using lower temperature water. The spray time was 15 seconds, the dwell time was 2 minutes, and the flow rate was 1.16 gpm (4400 ml/min). The nozzle was a red, glass-filled polypropylene nozzle commercially available from Spray Systems Co. and having a flow rate of 80 gpm at 10 psi and a 120 degree spray angle. The nozzle was placed about 1-1.5 inches from the solid product. The test results are presented in the histogram of FIG. 2 which plots the grams per gallon (g/gal) dispensed versus the temperature of the sprayed water in degrees Fahrenheit. The mean concentration was 8.97 g/gal.

### Example 70

The raw materials identified in Table 13 were mixed and formed into a solid product as described above with respect to Examples 1-5.

TABLE 14

| | Ex. 70 |
|---|---|
| De-ionized water (wt %) | 10% |
| Hexylene glycol (wt %) | 15% |
| Sodium stearate T-1 (wt %) | 5% |
| Tomadol 25-12 (wt %) | 36.63% |
| Tomadol 25-7 (wt %) | 14.09% |
| Citrus basil apple fragrance oil (wt %) | 19.28% |

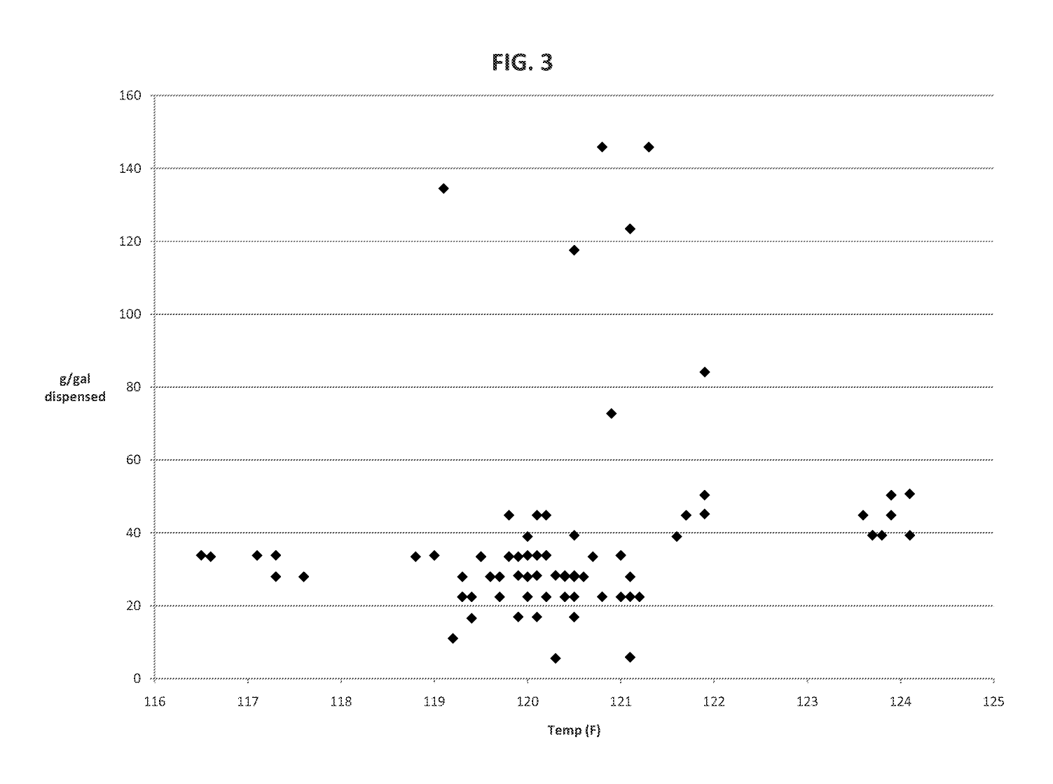

The solid product of Example 70 was tested in the load cell spray device as described above with respect to Example 68. The spray time was 15 seconds, the dwell time was 2 minutes, and the flow rate was 1.16 gallons per minute (gpm) (4400 milliliters per minute). The nozzle was a red, glass-filled polypropylene nozzle commercially available from Spray Systems Co. and having a flow rate of 80 gpm at 10 psi and a 120 degree spray angle. The nozzle was placed about 1-1.5 inches from the solid product. The test results are presented in the graph of FIG. 3 which plots the grams per gallon (g/gal) dispensed versus the temperature of the sprayed water in degrees Fahrenheit. The mean concentration was 38.73 g/gal.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the above described features.

1. A solid composition for cleaning or fragrancing, the solid composition comprising:
at least one ethoxylated alcohol;
at least one C12-C24 saturated fatty acid sodium salt;
water,
at least one glycol; and
at least one water insoluble component selected from a group consisting of fragrance oils, hydrocarbon solvents, glycol ethers, ester solutions, solvents, biocides, defoamers, lubricants, rinse additives and polishes, wherein a weight ratio of water to glycol to C12-C24 saturated fatty acid sodium salt is from about 2:2.5:1 to about 3:4:1.

2. The solid composition of claim 1, wherein the water insoluble component is less than about 10% soluble in about 20° C. water.

3. The solid composition of claim 1, wherein the at least one ethoxylated alcohol includes an ethoxylated C10-C15 alcohol.

4. The solid composition of claim 3 wherein, the at least one ethoxylated alcohol further includes a second ethoxylated C10-C15 alcohol.

5. The solid composition of claim 1, wherein the at least one glycol includes a member selected from group consisting of hexylene glycol and propylene glycol.

6. The solid composition of claim 1, wherein the water insoluble component constitutes between about 1% and about 30% by weight of the solid composition.

7. The solid composition of claim 1, wherein the C12-C24 saturated fatty acid sodium salt includes sodium stearate.

8. The solid composition of claim 1, wherein the solid composition consists essentially of:
at least one ethoxylated alcohol;
at least one C12-C24 saturated fatty acid sodium salt;
water;
hexylene glycol; and
at least one fragrance oil.

9. A hydrate solid comprising:
at least one ethoxylated alcohol;
at least one C12-C24 saturated fatty acid sodium salt;
water;
at least one glycol; and
at least one water insoluble component, wherein the water insoluble component is a fragrance oil or a mixture of fragrance oils, and wherein the hydrate solid is completely soluble in water having a temperature between about 50° F. and about 100° F.

10. The hydrate solid of claim 9, wherein the hydrate solid is stable when heated at about 50° C. for about 7 days.

11. The hydrate solid of claim 9, wherein the hydrate solid is completely soluble in water having a temperature between about 50° F. and about 100° F. when a water dilution ratio is at least 1%.

12. The hydrate solid of claim 9, wherein the at least one ethoxylated alcohol includes a first ethoxylated C10-C15 alcohol and a second ethoxylated C10-C15 alcohol, and the glycol is hexylene glycol.

13. The hydrate solid of claim 9, wherein the fragrance oil constitutes between approximately 1% and about 30% weight of the hydrate solid.

14. A method of making a water-dilutable solid cast composition for cleaning or fragrancing, the method comprising:
mixing solidification components and at least one water insoluble component to form a mixture, wherein the solidification components include at least one ethoxylated alcohol, at least one C12-C24 saturated fatty acid sodium salt, water, and at least one glycol, and wherein the water, glycol and C12-C24 saturated fatty acid sodium salt are present in a water to glycol to C12-C24 saturated fatty acid sodium salt weight ratio of between about 2:2.5:1 to about 3:4:1;
pouring the mixture into a casting; and
hardening the mixture to form a water-dilutable solid cast composition.

15. The method of claim 14, wherein the water insoluble component constitutes between approximately 1% and about 30% weight of the water-dilutable solid cast composition.

16. The method of claim 14, wherein the at least one ethoxylated alcohol includes a first ethoxylated C10-15 alcohol and a second ethoxylated C10-C15 alcohol, the C12-C24 saturated fatty acid sodium salt includes sodium stearate, and the glycol is hexylene glycol.

17. The method of claim 14, wherein the water-dilutable solid cast composition is completely soluble in water having a temperature between about 50° F. and about 100° F.

18. A method of forming a use solution, the method comprising:
dissolving solid detergent in water to form a use solution having a dilution ratio of at least 100:1, wherein the solid detergent comprises at least one ethoxylated alcohol, at least one C12-C24 saturated fatty acid sodium salt, water, at least one glycol, and at least one organic water insoluble component selected from a group consisting of fragrance oils and mixtures of fragrance oils, and wherein a weight ratio of water to glycol to C12-C24 saturated fatty acid sodium salt is from about 2:2.5:1 to about 3:4:1.

19. The method of claim 18, wherein dissolving the solid detergent with water to form a use solution comprises:
dissolving the solid detergent in the water to form a pre-dilution solution; and
diluting the pre-dilution solution with water to form a use solution.

20. The method of claim 18, wherein the at least one water insoluble component is less than about 10% soluble in about 20° C. water.

21. The method of claim 18, wherein the diluting step comprises:
dispensing the use solution at a rate of 1 gallon per minute, wherein the fragrance oil comprises at least about 0.2% by weight of the use solution.

22. The method of claim 18, wherein the use solution is a low viscosity liquid.

23. The method of claim 22, wherein the use solution has a viscosity less than about 100 centipoise.

24. The method of claim 18, wherein the at least one ethoxylated alcohol includes a first ethoxylated C10-C15 alcohol and a second ethoxylated C10-C15 alcohol.

25. The method of claim 18, wherein the glycol is hexylene glycol.

26. The method of claim 18, wherein the at least one water insoluble component concentration of the use solution is greater than a water solubility of the at least one water insoluble component in water at the same conditions.

27. The method of claim 18, wherein the use solution has a turbidity of less than 50 Nephelometric Turbidity Units (NTU).
